(19) United States
(12) Patent Application Publication
(10) Pub. No.: US 2025/0325170 A1
(43) Pub. Date: Oct. 23, 2025

(54) FILTER CHANGING APPARATUS FOR AN ENDOSCOPIC CAMERA, CAMERA HEAD FOR AN ENDOSCOPE, AND RETROFIT KIT FOR RETROFITTING A CAMERA HEAD AND/OR AN ENDOSCOPE

(71) Applicant: KARL STORZ SE & Co. KG, Tuttlingen (DE)

(72) Inventors: Maximilian KIMMI, Tuttlingen (DE); Kai KROLL, Tuttlingen (DE); Florian HUBER, Tuttlingen (DE)

(21) Appl. No.: 19/182,704

(22) Filed: Apr. 18, 2025

(30) Foreign Application Priority Data
Apr. 18, 2024 (DE) 10 2024 110 879.7

(51) Int. Cl. A61B 1/00; A61B 1/04

(52) U.S. Cl. CPC A61B 1/00186; A61B 1/00131; A61B 1/042; A61B 2560/0462

(57) ABSTRACT

The invention relates to a filter changing apparatus for an endoscopic camera, the filter changing apparatus having at least a first rotatable filter wheel, a second rotatable filter wheel and a rotatable first connecting wheel, the first filter wheel and the second filter wheel each having at least two receptacles for one optical filter each, the filter wheels having a common first rotary axle, the second filter wheel having a first engagement level with teeth distributed over the entire outer circumference thereof and the first connecting wheel being designed as a gear with teeth distributed over the entire outer circumference thereof, wherein the first filter wheel has a portion comprising teeth distributed along the outer circumference thereof, and the first connecting wheel is arranged on the outer circumference of the first filter wheel and on the outer circumference of the second filter wheel such that the teeth of the portion of the first filter wheel can mesh with the teeth of the first connecting wheel for rotating the first connecting wheel, and the teeth of the first connecting wheel can mesh with the teeth of the first engagement level of the second filter wheel so that when the first filter wheel is driven by means of the drive unit, at least one receptacle of each of the first filter wheel and the second filter wheel can be positioned in parallel in the optical passage. Furthermore, the invention relates to a camera head for an endoscope and to a retrofit kit for retrofitting a camera head and/or an endoscope.

### CROSS-REFERENCE TO RELATED APPLICATIONS

This application claims the benefit Under 35 U.S.C. 119(a) to German Patent Application No. 10 2024 110 879.7, filed Apr. 18, 2024, the disclosure of which is incorporated herein by reference in its entirety.

### SUMMARY

The invention relates to a filter changing apparatus for an endoscopic camera, wherein the filter changing apparatus has a base plate, an optical passage comprising an optical axis, at least a first rotatable filter wheel, a second rotatable filter wheel and a rotatable first connecting wheel as well as a drive unit for driving one of the filter wheels, wherein the first filter wheel and the second filter wheel each have at least two receptacles for one optical filter each, the filter wheels have a common first rotary axle, the second filter wheel has a first engagement level comprising teeth distributed over the entire outer circumference thereof, and the first connecting wheel is designed as a gear having teeth distributed over the entire outer circumference thereof. Furthermore, the invention relates to a camera head for an endoscope and to a retrofit kit for retrofitting a camera head and/or an endoscope.

In medical and non-medical applications, observation instruments such as endoscopes are used to examine internal cavities of a human or animal body or of an industrial, technical item, such as a pipeline. For imaging, a camera head having an image sensor can be used together with the endoscope. In order to improve the image quality and/or make different observation modes possible, it is known to introduce different filters into the beam path of the observation instrument.

For example, in fluorescence imaging, the item to be examined is exposed to light with an excitation radiation, which excites a fluorophore, which has been previously applied to the item or is already present, to emit light of a certain emission wavelength, wherein the excitation wavelength and the emission wavelength are usually different. Normally, the emission wavelength is longer than the excitation wavelength. The emitted emission light is usually significantly weaker than other light sources, such as excitation fluorescence light or imaging white light. For these reasons, it is necessary to filter out unwanted wavelength bands by means of a filter so that, if possible, only the desired spectrum and/or the emission wavelength of the fluorophore reaches the camera head in fluorescence mode. In addition to switching between different observation modes in which two or more filters are usually introduced one after the other into the beam path of the observation optics, it can also be advantageous to introduce two different filters into the beam path at the same time. In principle, different filter changers are known for this purpose.

DE 10 2020 100 676 B3 discloses a filter changing apparatus for an optical observation instrument having two beam paths, which comprises three filter wheels that are arranged one behind the other along a common axle and are rotatable around this common axle and relative to one another, wherein each filter wheel has at least one filter and at least one free optical passage so that a filter or a free optical passage of each filter wheel can be introduced into each of the two beam paths. The second filter wheel is drivable, and the first filter wheel is coupled to the second filter wheel via a first driver, and the third filter wheel is coupled to the second filter wheel via a second driver. The disadvantage here is that, due to the second driven filter wheel being in the middle and the connection of the first and third filter wheels arranged on both sides via a driver, the number of filter wheels that can be arranged in series one behind the other is limited to three.

Furthermore, a mechanical binary counter is known as an art object from the “Anstalt für freie Kunstmaschinen/Felix Scharstein” (www.scharstein.de/#binaerzaehler). This binary counter is based on a purely binary mechanism in which exactly two positions are provided for each revolution of a gear driven by a shaft. The number boards of the mechanical binary counter are arranged along a rotary axle with gears arranged therebetween, wherein these gears mesh in a partially transmitting manner with a second row of gears arranged therebehind on a second rotary shaft. The opposite board surfaces are arranged along the rotary axle and thus take up a lot of space in the longitudinal direction of the rotary axle. In addition, this mechanical binary counter requires a large number of individual parts, making the mechanism prone to failure.

The object of the invention is to improve upon the prior art.

The object is achieved by a filter changing apparatus for an endoscopic camera, the filter changing apparatus having a base plate, an optical passage comprising an optical axis, at least a first rotatable filter wheel, a second rotatable filter wheel and a rotatable first connecting wheel as well as a drive unit for driving one of the filter wheels, the first filter wheel and the second filter wheel each having at least two receptacles for one optical filter each, the filter wheels having a common first rotary axle, the second filter wheel having a first engagement level with teeth distributed over the entire outer circumference thereof and the first connecting wheel being designed as a gear with teeth distributed over the entire outer circumference thereof, wherein the first filter wheel has a portion comprising teeth distributed along the outer circumference thereof, and the first connecting wheel is arranged on the outer circumference of the first filter wheel and on the outer circumference of the second filter wheel such that the teeth of the portion of the first filter wheel can mesh with the teeth of the first connecting wheel for rotating the first connecting wheel, and the teeth of the first connecting wheel can mesh with the teeth of the first engagement level of the second filter wheel so that when the first filter wheel is driven by means of the drive unit, at least one receptacle of each of the first filter wheel and the second filter wheel can be positioned in parallel in the optical passage.

A filter changing apparatus is accordingly provided by means of which two or more filters can be precisely and efficiently pivoted into and out of the beam path of an endoscopic camera and/or an endoscope in parallel and at the same time. It is particularly advantageous that only a single drive unit is required for driving, this one drive unit also only acts on one filter wheel and causes this filter wheel to rotate, whereby by coupling the driven filter wheel to the adjacent filter wheel via the connecting wheel, the adjacent filter wheel itself can also be made to rotate as long as the associated teeth mesh with one another. Since the at least two filter wheels are arranged in series along the common rotary axle and thus with their respective opposing wheel surfaces transverse to the rotary axle, the filter wheels can be rotated clockwise and counterclockwise as desired in a fully transmitting and/or partially transmitting manner, whereby the optical filters to be pivoted-in in parallel can be combined as desired in the respective the receptacles. Above all, the simple clockwise and/or counterclockwise rotation allows for any sequence and rapid switching between different filters and/or filter combinations, and thus enables different observation modes and imaging in quick succession.

By sequentially arranging the filter wheels along the rotary axle, with their wheel surfaces arranged transversely to the rotary axle, a compact size of the filter changing apparatus is provided. Due to its compact size, the filter changing apparatus can be easily integrated into a camera head or used as a compact connector between a camera head and an endoscope. The dimensions of the filter changing apparatus are not only minimized in the longitudinal direction by sequentially arranging the filter wheels with their smaller material thickness along the rotary axle, but can also be reduced in the transverse direction. Although the filter wheels must have a sufficient diameter for the desired number of optical filter receptacles, a respective connecting wheel only has the function of transferring the rotational movement of the previous filter wheel to the next filter wheel. This means that the diameter of a respective connecting wheel can be significantly smaller than the diameter of the respective filter wheels so that the overall diameter of the filter changing apparatus can be minimized.

Since the first filter wheel and/or a terminal filter wheel has a portion having teeth distributed along the outer circumference thereof, when this filter wheel is driven, the connecting wheel is made to rotate by the meshing of the teeth of this first portion of the first filter wheel with the teeth of the first connecting wheel. The teeth of the first connecting wheel in turn mesh with the teeth of the first engagement level of the second filter wheel comprising teeth distributed over the entire outer circumference thereof. Thus, as a result of the portion comprising the teeth, upon a full revolution of the first filter wheel, the first connecting wheel causes half a revolution of the second filter wheel. This mechanical, originally binary mechanism allows different combinations of optical filters to be simultaneously pivoted into and arranged in the beam path using only a single drive unit. One optical filter per gear can be pivoted in, and accordingly at least two optical filters can be pivoted into and positioned in the beam path at the same time and in parallel.

An essential concept of the invention is based on the fact that when filter wheels are arranged sequentially, only the first and/or terminal filter wheel is drivable or driven and, as a result of a portion comprising teeth which does not take up the entire outer circumference of the first filter wheel, the rotational movement is only transmitted to the following second filter wheel via the connecting wheel when the circumferential teeth of a connecting wheel mesh with this portion, and thus the second filter wheel also rotates, and a corresponding filter receptacle of the second filter wheel can be pivoted into the beam path in parallel. When the circumferential portion free from the first portion having the teeth of the first filter wheel is aligned with the first connecting wheel, there is no engagement between the first filter wheel and the first connecting wheel, and the first connecting wheel and consequently also the second filter wheel remain stationary. Thus, a mechanical binary transmission mechanism is realized in which, when the first and/or previous filter wheel is rotated, the second and/or subsequent filter wheel either remains stationary or is rotated itself. As a result, a receptacle of each of the at least two filter wheels is arranged in the beam passage, and thus at least two filters are pivoted into the beam passage in parallel. Consequently, a filter apparatus is provided with which at least two identical and/or different filters, in particular fluorescence filters, can be pivoted into and out of the beam path of an endoscopic camera precisely, quickly, efficiently and especially at the same time with a long service life due to the purely mechanical transmission mechanism. In addition to the quick filter change, the frequency of use of a particular filter can also be specified by inserting the same and/or different filters into the receptacles. For example, an observation mode with normal white light is usually used more often than fluorescent light. Advantageously, a white light filter can thus be arranged upstream and/or downstream of a fluorescence filter in successive receptacles of a filter wheel or offset between the receptacles of two or more filter wheels so that, in addition to the desired more frequent use of white light by the respective change to a white light filter, the user can also clearly visually see the change between two other identical or different consecutive filters, for example two fluorescence filters. This reduces the risk of inadvertently working in the wrong observation mode.

The following terminology is explained:

A “filter changing apparatus” is in particular an apparatus with which at least two filters can be moved in parallel and at the same time into or out of the optical beam path and/or arranged in the optical passage. The filter changing apparatus can be activated in particular manually or automatically by means of a drive unit in order to change at least two filters in parallel by rotating the first and/or a terminal filter wheel. This means that one filter or two or more filters can be pivoted into and out of the optical passage automatically or manually by being rotated. The filter changing apparatus has at least a first filter wheel and a second filter wheel as well as at least a first connecting wheel, wherein the circumferential teeth of the first connecting wheel can mesh both with the teeth of the portion of the first filter wheel and the teeth of the first engagement level of the second filter wheel. Accordingly, the connecting wheel is preferably arranged on the outer circumference of the first filter wheel and the second filter wheel. The compact filter changing apparatus can in particular be integrated in a camera or can be connectable to the camera and/or an endoscope as a separate apparatus, for example designed as a snap-on filter. For automatically changing the filter, the filter changing apparatus may have an operating element, such as a switch, on its outer surface. Alternatively or in addition to a visual recognition of the filter change by the user, the filter changing apparatus can also have a display element and/or a sensor, for example a Hall effect sensor.

A “base plate” is in particular a component of the filter changing apparatus on and/or at the inside of which the at least one rotary axle is arranged. A base plate may also be a housing part and/or a housing cover of the filter changing apparatus.

A “filter wheel” (also called a “changing disc”) is in particular a disc which has at least two receptacles, each for an optical filter. The filter wheel can in particular be designed as a gear. In particular, the receptacles are evenly arranged relative to one another along an inner circumference of the filter wheel and/or distributed over the wheel surface of the filter wheel. The respective filter wheel is in particular rotatable around the rotary axle. The first filter wheel and/or one of the two terminal filter wheels can in particular be driven and rotated by hand and/or by means of a drive unit and/or a motor. For this purpose, the outer circumferential surface may, for example, be driven as a contact surface in that a drive unit and/or a gearbox acts at and/or on this contact surface. For example, the drive engages in an additional level of the first filter wheel and/or the terminal filter wheel which comprises teeth the entire way around in addition to the first portion in another level. Preferably, however, the first filter wheel and/or the terminal filter wheel is/are connected to a rotary shaft and driven by rotating the rotary shaft by means of a drive unit or by hand. The respective filter wheel is in particular a flat, planar component, the opposite wheel surfaces of which are substantially perpendicular to the optical axis and/or the rotary axle. Thus, the opposite wheel surfaces of at least two filter wheels or a plurality of filter wheels are aligned with one another and in particular transversely to the rotary axle. As a result, the filter wheels are arranged sequentially with their respective material thicknesses along the rotary axle. The respective filter wheel is designed in particular as a round wheel and/or disc. Preferably, the respective filter wheel is circular in cross section. However, the filter wheel can also have an elliptical cross-sectional shape and thus be designed as an elliptical wheel.

The respective filter wheel can have teeth and/or a gear rim distributed all the way around its outer circumference or has only a portion having teeth distributed along its outer circumference. The evenly distributed teeth of a filter wheel can be homogeneously and continuously formed along the material thickness of the filter wheel and thus along the optical axis and/or rotary axle, or the teeth are arranged in one level, and thus the teeth are not formed over the entire material thickness of the gear. Thus, a respective filter wheel can have teeth evenly distributed over its entire circumference and its entire material thickness. Likewise, a filter wheel can have teeth evenly distributed over a portion of its outer diameter and over its entire material thickness. A respective filter wheel can also have teeth evenly distributed along its entire outer diameter in a first engagement level and have a portion comprising the evenly distributed teeth on its end face and/or outer surface in a second engagement level along the rotary axle and/or optical axis, wherein the teeth of the first engagement level and the second engagement level comprising the portion are designed in particular to be the same and/or continuous along the material thickness. The portion of the outer surface of the respective gear and/or the respective engagement level which is free of teeth has, in this region free of teeth, in particular a smaller diameter than a portion and/or an engagement level comprising distributed teeth. Thus, the portion along the outer circumference free of teeth can be designed as a recess and/or cutout. Thus, a respective filter wheel with a first engagement level can be designed as a complete gear and a second engagement level can be designed as a partial gear.

In particular, the filter wheels each have a bore passing partially or completely through their material thickness for the insertion and/or passage of the first rotary axle. The respective bore is in particular arranged centrally relative to the diameter of the respective filter wheel. The first rotary axle for the filter wheels is in particular fastened inside the housing of the filter changing apparatus to the base plate and/or a housing cover thereof. Since the optical passage through the housing of the filter changing apparatus is preferably arranged centrally relative to the outer diameter of the housing, the first rotary axle of the filter wheels and/or the second rotary axle of the connecting wheel or connecting wheels is/are correspondingly arranged further outward and off-center. The first filter wheel and/or a terminal filter wheel can in particular have a shaft for driving, wherein the shaft is arranged on the side of the wheel surface that is opposite the side aligned with the second filter wheel.

By forming the first filter wheel with a portion comprising distributed teeth and meshing these teeth with the teeth of the first connecting wheel, only a partial rotation is transmitted by means of the first connecting wheel to the second filter wheel, which is also in engagement with the first connecting wheel upon a complete revolution of the first filter wheel. Thus, the first filter wheel and/or a terminal filter wheel in particular is only partially transmissive to the first connecting wheel and the coupled second filter wheel. The maximum rotation distance for one full revolution of the filter wheel to achieve the desired filter combination is (2ⁿ)/2 where n=number of filter wheels.

A “connecting wheel” (also “auxiliary gear”) is in particular a gear. The connecting wheel has in particular teeth evenly distributed over the circumference and/or the material thickness thereof. The connecting wheel is designed in particular as a spur gear and/or cylindrical gear. In particular, the rotary axle of the connecting wheel is parallel to the rotary axle of the filter wheels. The respective connecting wheel and the two filter wheels into which this connecting wheel engages form, in particular, spur gearing. In particular, a connecting wheel has a significantly smaller diameter than the diameter of a filter receptacle. Two or more connecting wheels can be arranged on a common rotary axle. For this purpose, the respective connecting wheel has in particular a central through-bore for the passage of the rotary axle. The rotary axle can be fastened to the inside of the housing of the filter changing apparatus, for example on a base plate or a housing cover.

The “first rotary axle” is designed in particular as a common axle for the rotatable filter wheels, and the “second rotary axle” is designed as a common axle for the connecting wheels. A “common rotary axle” is understood to mean, in particular, a machine element for supporting the rotatable filter wheels or connecting wheels. In particular, the common rotary axle itself does not rotate and therefore does not transmit any torque. The fixed common rotary axle thus also represents a common rotary axle for the filter wheels or the connecting wheels. The common rotary axle also ensures, in particular a sequential arrangement of the filter wheels or the connecting wheels along the common rotary axle. For this purpose, each filter wheel preferably has a central bore so that the common rotary axle is guided through the central bores of the filter wheels, and thus the filter wheels lie “in alignment” in a straight line with their center points.

In principle, it should be pointed out that the terms “first” and “second” filter wheel or “first” and “second” portion, filter and other terms only serve for differentiation purposes and do not necessarily specify a sequence.

A “receptacle” (also called a “filter receptacle”) is in particular a cavity and/or a hollow body that can be inserted into an opening within the respective filter wheel. The receptacle can in particular have an external partial or complete enclosure and/or border into which an optical filter can be inserted and which at least partially encloses the optical filter at its circumference. A receptacle may, for example, be a short tubular body. The receptacle in particular forms a protective casing for the optical filter. However, the receptacle can also be designed directly as a continuous opening, for example as a bore, through the material thickness of the respective filter wheel. Optionally, the respective filter wheel can also have a filter receptacle which does not comprise an optical filter and thus allows free passage through the optical beam path. Accordingly by means of the respective filter wheel, an empty filter receptacle can also be introduced into the optical passage and into the beam path. Likewise, instead of omitting an optical filter, free passage can also be made possible by a non-filtering optical element, such as a glass pane. A glass pane used as a window can also have an anti-reflective coating.

An “optical filter” (also simply referred to as a “filter”) is in particular an optical element which selects the incident radiation and/or incident beams on the basis of specific properties, such as wavelength, polarization state, angle of incidence and/or direction of incidence, and thus allows it and/or them to pass through or prevents it and/or them from passing through. Likewise, an optical filter can change the properties of the light passing through, for example by converting circularly polarized light into linearly polarized light. In particular, an optical filter can block a specific spectral wavelength band. An optical filter may, for example, be a graduated filter, an edge filter, a polarizing filter or an interference filter. An interference filter in particular has a coating which blocks light of a certain spectral range or allows it to pass through. The optical filter can in particular be used as an observation filter and/or detection filter, fluorescence observation filter or excitation filter. The optical filter in particular comprises glass or a crystalline material. The optical filter may be planar or designed as a filter lens. In principle, instead of the optical filter, another optical element, such as a lens, an aperture, a polarizer or a similar optical element, can also be arranged in the filter changing apparatus and/or the filter receptacle.

A “white light filter” is understood in particular to mean that a corresponding receptacle and/or position is free of an optical element, or an optical element in a corresponding receptacle and/or position is free of a filter function so that the light and/or white light is allowed to pass through, in particular unchanged. White light is let through by a white light filter without changing its light properties, especially the wavelengths. A white light filter can also be a filter that blocks near-infrared light. A “white light filter” can also be a filter that filters the light to improve the quality of the image when illuminated with white light. For this purpose, a BG39 filter from Schott can be used, for example. Thus, by means of a white light filter, the light captured by an image sensor and/or a camera can also be adapted to a sensitivity curve and/or a specific sensitivity of the human eye.

A “fluorescence observation filter” (also referred to as a “fluorescence filter”) is in particular an optical polychroitic interference filter for separating the emitted fluorescent light from the excitation light used. The fluorescence filter thus blocks the specific fluorescence excitation radiation and allows the fluorescence emission radiation to pass along the optical beam path.

Preferably, the fluorescence filter completely blocks the excitation light while allowing the fluorescence emission light to pass through, which usually has a longer wavelength than the excitation light. Thus, a fluorescence filter is in particular an observation filter that filters out the excitation light that causes a fluorophore to glow. This is advantageous because the excitation light is usually several orders of magnitude brighter than the resulting and/or emitted fluorescent light and would otherwise outshine it. A fluorescence filter can also be a “blue filter”, “red filter”, “IR filter” or “NIR filter”.

A “blue filter” is understood in particular to mean a filter which filters out the blue excitation light from a light source, but at least predominantly allows the fluorescent light, in particular fluorescent light emitted by a fluorophore, to pass through. For example, when using the fluorophore FITC (fluorescein isothiocyanate, green derivative of fluorescein), excitation occurs with an LED at a wavelength of 460 nm, wherein longer-wavelength fluorescent light with a maximum of approximately 520 nm in the green spectral range is emitted by the fluorophore. In order to be able to clearly display the fluorescent light emitted by the FITC during imaging, the blue excitation light is filtered out by the filter changing apparatus and/or camera.

A “red filter” is understood in particular to mean a filter which filters out the red excitation light from a light source, but at least predominantly allows the fluorescent light, in particular fluorescent light emitted by a fluorophore, to pass through.

An “IR filter” is understood to mean in particular a filter which filters out infrared excitation light from a light source, but at least predominantly allows the fluorescent light, in particular fluorescent light emitted by a fluorophore, to pass through.

An “NIR filter” is understood in particular to mean a filter which filters out near-infrared excitation light from a light source, but at least predominantly allows the fluorescent light, in particular fluorescent light emitted by a fluorophore, to pass through.

An “optical passage” is in particular a hollow space in the filter changing apparatus through which light can pass. An optical passage is in particular a continuous opening through the filter changing apparatus, the additional components thereof and/or a housing of the filter changing apparatus. The optical passage is in particular arranged around the center of the cross section of the rotatable filter wheels and/or around the optical axis. The optical passage extends in particular along the optical axis. In particular, a filter receptacle and/or an optical filter can be arranged in front of and/or in the optical passage in the light propagation direction. Likewise, when light passes through, the optical passage may be free of an arranged optical filter and/or a receptacle. In principle, the optical passage can have any cross-sectional shape, but the optical passage is preferably circular in cross section.

An “optical axis” is in particular a line along which a degree of rotational symmetry exists in an optical system. The optical axis is in particular an imaginary line that defines a path along which light propagates through the filter changing apparatus and/or the camera toward an image sensor. Preferably, the optical axis runs through the curvature means of the particular pivoted-in filter and/or of a downstream lens system and/or objective system. However, the optical axis can also be bent and/or directed by a lens, an optical element and/or one of the optical filters. The optical beam path as a geometric course of light beams is in particular arranged in and/or around the optical axis and runs along, converges toward and/or disperses from the optical axis.

A “camera” (also called a “camera head”) is in particular a piece of equipment for receiving image light along an optical axis from an endoscope and for focusing the received image light on at least one image sensor. In addition to the at least one image sensor, the camera may in particular have an aperture or a window for letting through the received image light, and a lens system for focusing the image light on the at least one image sensor. The image data recorded by at least one image sensor can in particular be transmitted electronically by the camera head to a display system and/or to an image processing unit in order to display the endoscopic image to the user. The camera may have means for detecting the connected endoscope and for processing algorithms. A connector for connecting an endoscope to the camera can be arranged at the distal end of the endoscope and/or the proximal end of the camera head. The filter changing apparatus according to the invention itself may also be designed as a connector for connecting an endoscope to a camera. To avoid vignetting and thus shadowing at the edge of the image, the filter changing apparatus is preferably integrated into the camera module.

An “endoscope” is in particular a medical or industrial piece of equipment for endoscopic examination and inspection of a human or animal body cavity and/or an industrial cavity, such as a tube. The endoscope in particular has a handpiece, a shaft, a light source, a light guide, a sensor and/or a camera. The endoscope is in particular a video endoscope, which has digital image recording and image transmission and thus an integrated or connectable camera. In addition to medical and veterinary applications, an endoscope and/or video endoscope may, however, also be used for industrial purposes, for example for visual inspection in hard-to-reach cavities. In industrial applications, an endoscope is often referred to as a borescope.

An “image sensor” is in particular a light-sensitive electronic component which is based on an internal photoelectric effect. By means of the image sensor, one or more images from the viewing area of the imaging apparatus are in particular recorded and converted into electronic signals. The image sensor has a sensor plane in the image plane of the optical system, of a lens system and/or of the objective. An electronic image sensor may in particular be a CCD sensor (charge-coupled device) or a CMOS sensor (complementary metal-oxide semiconductor).

In a further embodiment, the filter changing apparatus has a second connecting wheel, a third connecting wheel and/or optionally further connecting wheels and a third filter wheel, a fourth filter wheel and/or optionally further filter wheels, wherein the third filter wheel, the fourth filter wheel and/or optionally the further filter wheels each have at least one first engagement level with teeth distributed uniformly completely over the respective outer circumference.

Thus, the number of consecutive filter wheels along the first rotary axle can be scaled as desired, and the filter changing apparatus can be extended. Any number of intermediate wheels can thus be arranged between the driven first and/or terminal filter wheel and an opposite second terminal wheel. In particular, these intermediate wheels have at least one first engagement level with teeth evenly distributed completely over the respective outer circumference, with which the teeth of the connecting wheel, which also meshes with the teeth of the previous filter wheel, mesh. In order to get from an existing filter position to a new filter position when using the filter changing apparatus, a maximum of (2ⁿ)/2 revolutions of the first driven and/or the terminal filter wheel are necessary. In this case, the first driven and/or terminal filter wheel can be rotated in particular clockwise or counterclockwise. It is particularly advantageous that, despite an arbitrarily large number of filter wheels, little installation space is required along the rotary axle and/or the optical axis due to the sequencing of the filter wheels with their material thicknesses along the first rotary axle.

A third, fourth and optionally further filter wheel is in principle a filter wheel as defined above. However, these third, fourth and optionally further filter wheels are arranged in particular as intermediate wheels with a different arrangement of the teeth evenly distributed along the outer circumference. These third, fourth and optionally further filter wheels as intermediate wheels have in particular a first engagement level with teeth evenly distributed completely over the respective outer circumference. Preferably, all the teeth of the filter wheels and/or the connecting wheels have the same tooth geometry in order to ensure optimal transmission of the movement to the next connecting wheel and/or filter wheel. In principle, however, it should be noted that the respective teeth of the filter wheels and/or the connecting wheels can also have a different geometry, for example tooth flanks or meshing points, as long as a connecting wheel can mesh with the respective associated teeth of the filter wheels.

The terminal filter wheel, which is arranged opposite the first and/or terminally driven filter wheel and encloses, together therewith, the intermediate wheels arranged therebetween, can, instead of the at least one first engagement level comprising teeth uniformly distributed completely over the respective outer circumference, also have teeth which are formed over the entire material thickness of this terminal filter wheel.

A second, third and optionally further connecting wheel is in principle a connecting wheel as defined above. Preferably, all connecting wheels are geometrically identical. However, the connecting wheels may also differ slightly from one another in terms of their geometric shape as long as the respective connecting wheel performs its function of transmitting the rotational movement of the preceding filter wheel to the following filter wheel.

In order to continue the binary mechanical transmission mechanism to the subsequent filter wheel, the second filter wheel, the third filter wheel, the fourth filter wheel and/or optionally the further filter wheels may each have a second engagement level with a portion comprising teeth distributed along the respective outer circumference.

In a further embodiment of the filter changing apparatus, the portion of the first filter wheel and/or the portion of the respective second engagement level of the respective of the second filter wheel, the third filter wheel, the fourth filter wheel and/or optionally each further filter wheel has or have teeth in a region covering 45% to 55%, in particular 48% to 52%, preferably 50%, of the respective outer circumference.

Thus, for a full revolution of the preceding filter wheel, approximately or exactly half a revolution is transferred to the subsequent filter wheel by means of the associated connecting wheel, since the toothless portion of the first filter wheel and/or the respective engagement level of a filter wheel rotates past the associated connecting wheel without engagement and without contact.

In order to provide redundant filter combinations and thus enable fast switching between different combinations, the respective filter wheel can have four receptacles, six receptacles and/or optionally an even number of further additional receptacles for one optical filter each.

Due to an increased number of receptacles and thus spaces for optical filters on the respective filter wheel, certain filter combinations are made available multiple times when changing filters and continuing to rotate, which enables faster switching and changing between different filter combinations. In principle, proceeding from two filter receptacles per filter wheel, the number of filter receptacles of the filter wheel can vary as desired. However, an even number of receptacles is required since otherwise, due to the mechanics, a filter of a subsequent filter wheel could be covered by the preceding filter wheel. More than two filter receptacles on the respective filter wheel are realized by the respective subsequent filter wheel only moving after a half rotation of 180° of the preceding wheel.

In a further embodiment of the filter changing apparatus, the drive unit has a single motor for driving the first filter wheel or a terminal filter wheel of the filter changing apparatus.

Due to the mechanical, binary translation and transmission of the movement of the first filter wheel or a terminal filter wheel designed as described above for the first filter wheel, only a single motor is required as the drive to move all the filter wheels by means of the connecting wheels. This increases the service life of the filter changing apparatus and reduces wear and installation space since each filter wheel does not have to be driven by its own motor. Due to the mechanical, binary transmission between the filter wheels, desired filter combinations can be achieved in parallel in the beam path with only one motor and can even be provided redundantly. The motor can drive a shaft connected to the first and/or terminal filter wheel.

To realize a drive over the outer circumference of the first and/or the terminal filter wheel, the first filter wheel or the terminal filter wheel can have a drive portion comprising teeth distributed completely over the respective outer circumference so that the first filter wheel or the terminal filter wheel can be driven via a drive gear by means of the one single motor.

In a further embodiment of the filter changing apparatus, the first filter wheel or the terminal filter wheel has a shaft or a receptacle for a shaft along the first rotary axle for connection to the one single motor.

In order to minimize the number of components of the filter changing apparatus, the connecting wheels can have a common second rotary axle.

In a further embodiment of the filter changing apparatus, the first rotary axle and/or the second rotary axle is or are arranged on the base plate.

In order to provide an exact holding mechanism during the period in which the respective filter wheel does not mesh with its teeth in a connecting wheel, the filter changing apparatus can have a holding device for holding a non-rotating filter wheel in its position during driving.

A “holding device” can in particular be a device and/or a component which ensures that a non-rotating filter wheel or plurality of filter wheels is/are held in their respective rest position. The holding device can be implemented, for example, by means of magnetic force, a spring, friction and/or another mechanism.

Thus, while a toothless portion of the respective filter wheel rotates past the associated connecting wheel without engaging, rotation of the subsequent filter wheel can be prevented by mechanically holding it in and/or at its position by means of the holding device, for example a resilient pressure piece. In addition, a spacer or other structural design can prevent direct, frictional contact between the wheel surfaces of the successive filter wheels.

In a further aspect of the invention, the object is achieved by a camera head for an endoscope, wherein the camera head has an image sensor, an opening for receiving light of an image along an optical path, and an optical lens system for focusing the light on the image sensor, wherein the camera head has at least one filter changing apparatus as described above.

This provides a camera head on or in which a compact, space-saving filter changing apparatus is arranged. If at least one filter changing apparatus is arranged directly in the camera head, the camera head can be detachably connected to different types of endoscopes. Of course, the camera head can also have two or more filter changing apparatuses in a row in an optical path and/or along the optical axis, even if a single filter changing apparatus already realizes a diverse selection of filter combinations, various possible filter settings and applications due to the parallel arrangement of any number of filter wheels and thus filters. Thus, the camera head enables different multispectral imaging and/or a broad application of different fluorophores in fluorescence imaging.

In order to adjust a currently used filter configuration or adapt it to desired different observation modes, the camera head may comprise a detection unit for detecting an identification of each optical filter in the optical path. Likewise, the camera head can have a control unit for adjusting the rotation speed of the filter plate and for checking and/or adjusting the particular optical filter, arranged in the beam path, according to the operating mode selected at the time.

In a further aspect of the invention, the object is achieved by a retrofit kit for retrofitting a camera head and/or an endoscope, wherein the retrofit kit comprises at least one filter changing apparatus as described above, so that the filter changing apparatus can be arranged between a proximal end of the endoscope and a distal end of the camera head.

This provides a retrofit kit (also referred to as an “adapter”) with at least one filter changing apparatus, which retrofit kit simultaneously serves both as a connector between an existing endoscope and an existing camera head and to make different observation modes possible. In addition, the retrofit kit may also comprise two or more filter changing apparatuses arranged in a row between the endoscope and the camera head, or a filter changing apparatus may be replaced by another filter changing apparatus according to the invention to make different applications and/or observation options possible.

### BRIEF DESCRIPTION OF THE DRAWINGS

The invention is explained in more detail below with reference to exemplary embodiments. In the figures:

FIG. 1 is a schematic three-dimensional detail view of an endoscope system with an endoscope, a filter changer and a camera head,

FIG. 2 is a three-dimensional representation of a filter changer in side view with an endoscope receptacle and a camera receptacle,

FIG. 3 is a three-dimensional view of the open filter changer from FIG. 2 with a view of the inside,

FIG. 4 is a three-dimensional view of a first filter gear,

FIG. 5 is a three-dimensional view of a second filter gear,

FIG. 6 is a three-dimensional view of a third filter gear,

FIG. 7 is a three-dimensional view of a connecting gear, and

FIG. 8 is a three-dimensional view of the filter changer with the housing open with the filter gears and the connecting gears.

### DETAILED DESCRIPTION

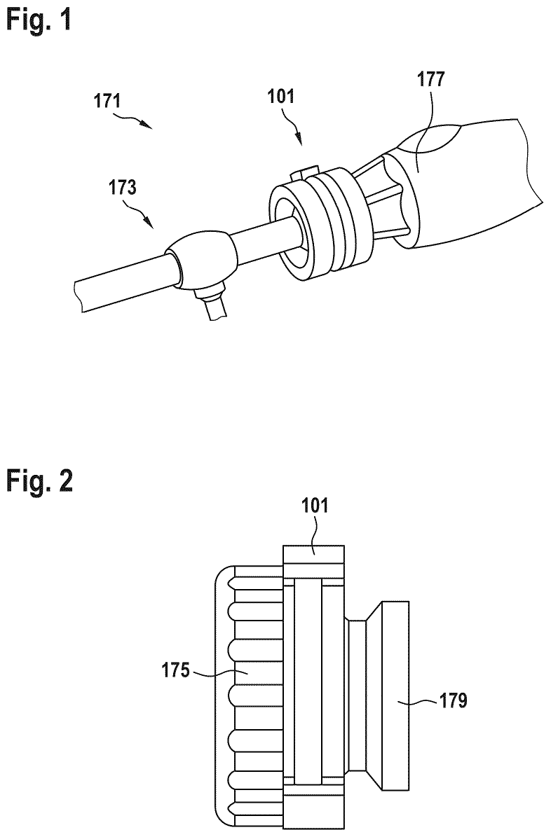

An endoscope system 171 has a camera head 177, a filter changer 101 and an endoscope 173. The filter changer 101 is connected to the camera head 177 by means of a camera receptacle 179 and to the endoscope 173 by means of an endoscope receptacle 175 (FIGS. 1 and 2). Furthermore, the filter changer 101 has a housing 103 with a base plate 107 designed as a distal cover. The base plate 107 is externally connected to a proximal cover of the housing 103 by means of screws 105.

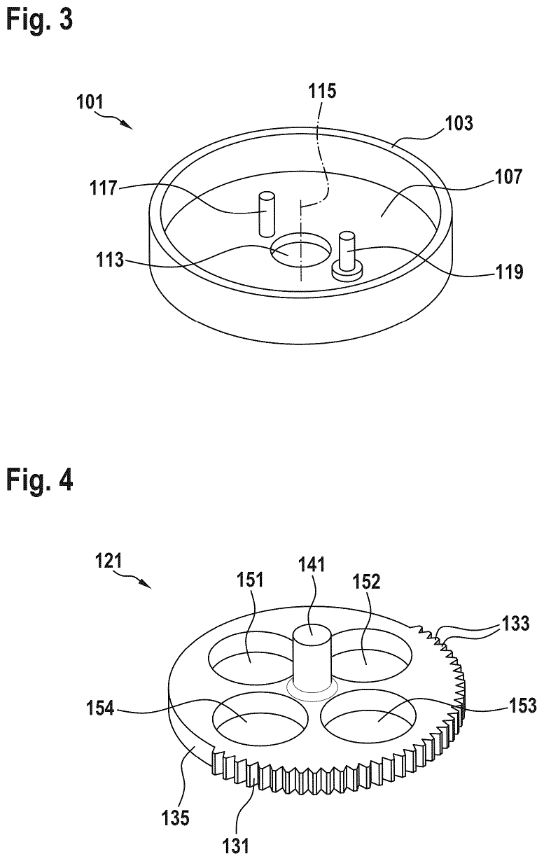

Inside the housing 103, the filter changer 101 has a first filter gear 121, a second filter gear 123 and a third filter gear 125, wherein the third filter gear 125 is arranged above the base plate 107. Furthermore, the filter changer 101 has a first connecting gear 127 and a second connecting gear 129 (FIG. 8). An optical passage 113 comprising an optical axis 115 is formed continuously through the base plate 107. The optical passage 113 and the optical axis 115 are arranged in the middle and centered with respect to an outer diameter of the base plate 107 and the housing 103. Inside the housing 103, the base plate 107 has a connected first rotary axle 117 and a second rotary axle 119 (FIG. 3).

The first filter gear 121 has a first filter receptacle 151, a second filter receptacle 152, a third filter receptacle 153 and a fourth filter receptacle 154 which are arranged at a distance from a central shaft 141 and an outer diameter of the first filter gear. Different optical filters (not shown) are inserted into these four filter receptacles 151, 152, 153 and 154. On its outer side, the first filter gear 121 has a portion 131 comprising teeth 133, which occupies 50% of the outer circumference of the first filter gear 121 and thus describes a circular arc of 180°. The other 50% of the outer circumference of the first filter wheel 121 is formed as a cutout 135 free of teeth. The shaft 141 has an internal bore for receiving the first rotary axle 117 on an underside and thus opposite the upper wheel surface shown in FIG. 4.

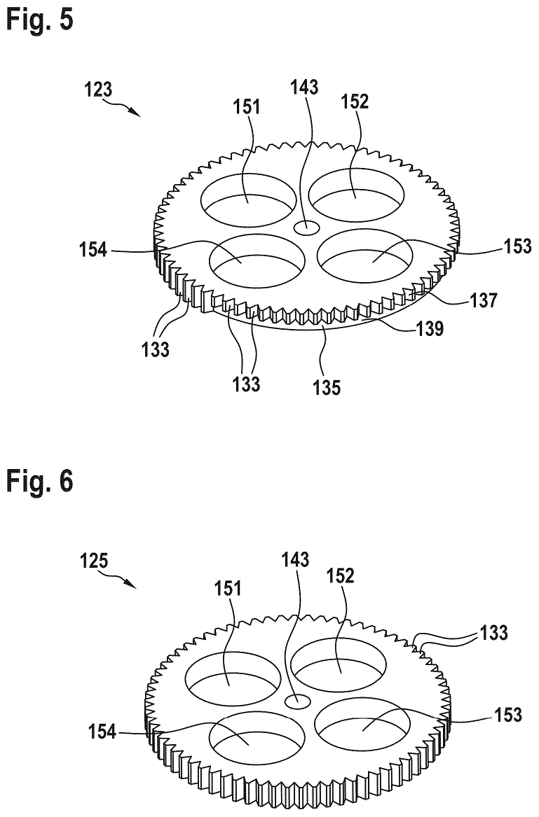

The second filter gear 123 also has four filter receptacles 151, 152, 153 and 154. In the center of the second filter gear 123, a continuous bore 143 for the first rotary axle 117 is formed. On its outer circumference, the second filter gear 123 has a first engagement level 137 with circumferential teeth 133 that adjoins the outside of the upper gear surface shown in FIG. 5. Below this first engagement level 137, a second engagement level 139 has a portion comprising a cut-out 135 along 50% of the outer circumference of the second filter gear 123, wherein the other half of the outer circumference is formed with continuous teeth 133 over the first engagement level 137 and the second engagement level 139.

The third filter gear 125 also has four filter receptacles 151, 152, 153 and 154 as well as a central bore 143 for receiving the first rotary axle 117. The third filter gear 125 has continuous teeth 133 all around and along its material thickness.

During a previous assembly process of the filter changer 101, the third filter gear 125 was placed with its bore 143 on the first rotary axle 117 so that the underside of the third filter gear 125 lies on the inner side of the base plate 107. Subsequently, with its upper side facing upward, the second filter gear 125, as shown in FIG. 5, was placed with its bore 143 on the first rotary axle 117 so that the underside of the second filter gear 123 lies on the upper side of the third filter gear 125. Finally, the first filter gear 121 was placed with its bore introduced inside of the shaft 141 on the first rotary axle 117 so that the underside of the first filter gear 121 lies on the upper side of the second filter gear 123. The first rotary axle 117 is arranged at a distance from the optical axis 115 and the optical passage 113 such that each of the four filter receptacles 151, 152, 153 and 154 can be pivoted into and arranged concentrically in the optical passage 113 and with respect to the optical axis 115.

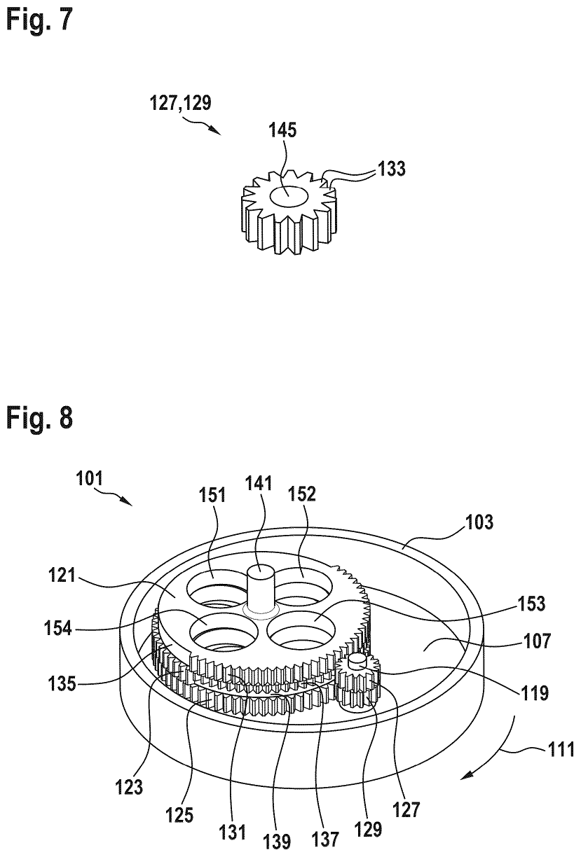

The first connecting gear 127 and the second connecting gear 129 are of the same design with a central bore 145 for the second rotary axle 119 and with teeth formed continuously over their outer diameter and height. The first connecting gear 127 is arranged on the second connecting gear 129 around the second rotary axle 119 (FIGS. 7 and 8). In this case, the teeth 133 of the first connecting gear 127 mesh with the teeth 133 of the first filter gear 121 and the second filter gear 123, while the teeth 133 of the second connecting gear 129 mesh with the teeth 133 of the third filter gear 125. Thus, the first connecting gear 127 combines the portion 131 with teeth 133 of the first filter gear 121 for motion transmission with the first engagement level 137 with fully circumferential teeth 133, and the second connecting gear 129 connects the second engagement level 139 of the second filter gear 123 with the portion comprising 50% of the surrounding teeth 133 to the teeth 133 of the third filter gear 125.

The following operations are carried out by means of the filter changer 101 and the endoscope system 171.

The shaft 141 is driven on the proximal side by means of a single motor (not shown in the figures), and thereby rotates the first filter gear 121 clockwise in the direction of rotation 111. As long as the teeth 133 of the first connecting gear 127 mesh with the teeth 133 of the portion 131 of the first filter gear 121, the first connecting gear 127 moves around the second rotary axle 119. If the first connecting gear 127 simultaneously meshes with the teeth 133 of the first engagement level 137 with the fully circumferential teeth 133 of the second filter gear 123, the second filter gear 123 is accordingly also rotated in the direction of rotation 111. Since all three filter gears 121, 123 and 125 are mounted only on the first rotary axle 117, the transmission of movement from the first driven filter gear 121 to the second filter gear 123 and the subsequent third filter gear 125 takes place only via the two connecting gears 127 and 129. Accordingly, during a complete revolution of the first filter gear 121 in the direction of rotation 111, as a result of the first portion 131 with teeth 133, the second filter gear 123 is rotated by half a revolution by means of the first connecting gear 127, since when the first connecting gear 127 is present in the region of the cutout 135 of the first filter gear 121, the teeth 133 of the first connecting gear 127 do not mesh. Thus, the first connecting gear 127 is not rotated during half a revolution of the first filter gear 121. During a full revolution of the second filter gear 123, the third filter gear 125 is also rotated by half a revolution by means of the second connecting gear 129 as long as the teeth 133 of the second connecting gear 129 mesh with the 50% of the circumferential teeth 133 of the second engagement level 139 of the second filter gear 123.

By each quarter rotation of the driven first filter gear 121, the first filter receptacle 151, the second filter receptacle 152, the third filter receptacle 153 and the fourth filter receptacle 154 are subsequently introduced into the optical passage 113. One filter A, B, C and D is inserted into each of the four filter receptacles 151 to 154 of the first filter gear 121. Likewise, these four filters A, B, C and D are inserted into the corresponding filter receptacles 151 to 154 of the second filter gear 123 and the third filter gear 125. This results in the filter combinations shown in Table 1. For a filter changer 101 as described above with the three filter gears 121, 123 and 125 and the four filter receptacles 151 to 154, all the filter combinations shown in Table 1 are possible. If the three filter gears 121, 123 and 125 each have only two filter receptacles, only the filter position combinations marked with a star in front can be realized.

TABLE 1
Possible ways of combining four filters A,
B, C, D of the filter gears 121, 123, 125

| | | | | Angle of rotation |
|---|---|---|---|---|
| | Filter gear 125 | Filter gear 123 | Filter gear 121 | of filter gear 121 |
| * | A | A | A | 0° |
| | A | A | B | 90° |
| * | A | A | C | 180° |
| | A | B | D | 270° |
| * | A | C | A | 360° |
| | A | C | B | 450° |
| * | A | C | C | 540° |
| | B | D | D | 630° |
| * | C | A | A | 720° |
| | C | A | B | 810° |
| * | C | A | C | 900° |
| | C | B | D | 990° |
| * | C | C | A | 1080° |
| | C | C | B | 1170° |
| * | C | C | C | 1260° |
| | D | D | D | 1350° |

In an alternative of the filter changer 101, the filter changer 101 only comprises the first filter gear 121 and the third filter gear 125 as terminal filter gears as well as a connecting first connecting gear 127. The four filter receptacles 151 to 154 of the first filter gear 121 and the third filter gear 125 are equipped with an example filter selection (Table 2). This alternative of the filter changer 101 comprising only the first filter gear 121 and the third filter gear 125 is in principle operated as described above. As a result, the filter combinations shown in Table 3 can be set by means of the driven rotation of the first filter gear 121. Thus, even with the alternative of the filter changer 101 with only the two filter gears 121 and 125, all potential filter combinations are possible and settable. However, as a comparison of the filter combinations in Table 3 with Table 1 shows, the filter changer described above comprising the three filter gears 121, 123 and 125 has the advantage that repeating, redundant filter combinations are provided, thereby reducing the length of a revolution and thus the switching distance and the changeover time between particularly important and frequently used filter combinations.

TABLE 2
Example of a possible filter selection in a
filter changer comprising two filter gears

| Filter gear | Filter A | Filter B | Filter C | Filter D |
|---|---|---|---|---|
| 125 | No filter | Red filter | White filter | NIR filter |
| 121 | White filter | Blue filter | No filter | No filter |

TABLE 3
Possible ways of combining four filters
A, B, C, D in two filter gears

| Filter gear 125 | Filter gear 121 | Combination |
|---|---|---|
| A | A | No filter, white filter |
| A | B | No filter, blue filter |
| A | C | No filter, no filter |
| B | D | Red filter, no filter |
| C | A | White filter, white filter |
| C | B | White filter, blue filter |
| C | C | White filter, no filter |
| D | D | NIR filter, no filter |

This provides a filter changer 101 with which at least two different filters can be quickly, efficiently and precisely pivoted in parallel into the optical beam path by means of the filter gears 121, 123, 125, and that can be used with different types of endoscopes 173 and camera heads 177.

### LIST OF REFERENCE SIGNS

101 filter changer
103 housing
107 base plate
111 rotation direction
113 optical passage
115 optical axis
117 first rotary axle
119 second rotary axle
121 first filter gear
123 second filter gear
125 third filter gear
127 first connecting gear
129 second connecting gear
131 portion comprising teeth
133 tooth
135 cutout
137 first engagement level
139 second engagement level
141 shaft
143 bore for first rotary axle
145 bore for the second rotary axle
151 first filter receptacle
152 second filter receptacle
153 third filter receptacle
154 fourth filter receptacle
171 endoscope system
173 endoscope
175 endoscope receptacle
177 camera head
179 camera receptacle

1. A filter changing apparatus for an endoscopic camera, the filter changing apparatus comprising:
a base plate,
an optical passage comprising an optical axis,
at least one first rotatable filter wheel,
a second rotatable filter wheel and a rotatable first connecting wheel as well as a drive unit configured to drive one of the filter wheels, the first filter wheel and the second filter wheel each having at least two receptacles for one optical filter each, the filter wheels having a common first rotary axle, the second filter wheel having a first engagement level with teeth distributed over the entire outer circumference thereof, and the first connecting wheel being designed as a gear with teeth distributed over the entire outer circumference thereof, wherein the first filter wheel has a portion comprising teeth distributed along the outer circumference thereof, and the first connecting wheel is arranged on the outer circumference of the first filter wheel and on the outer circumference of the second filter wheel in such a way that the teeth of the portion of the first filter wheel can mesh with the teeth of the first connecting wheel for rotating the first connecting wheel, and the teeth of the first connecting wheel can mesh with the teeth of the first engagement level of the second filter wheel so that when the first filter wheel is driven by means of the drive unit, at least one receptacle of the first filter wheel and of the second filter wheel can be positioned in parallel in the optical passage.

2. The filter changing apparatus according to claim 1, wherein the filter changing apparatus has a second connecting wheel, a third connecting wheel and/or optionally further connecting wheels and a third filter wheel, a fourth filter wheel and/or optionally further filter wheels, the third filter wheel, the fourth filter wheel and/or optionally each of the further filter wheels having at least one first engagement level with teeth distributed uniformly completely over the respective outer circumference.

3. The filter changing apparatus according to either claim 1, wherein the second filter wheel, the third filter wheel, the fourth filter wheel and/or optionally each of the further filter wheels has or have a second engagement level comprising a portion with teeth distributed along the respective outer circumference.

4. The filter changing apparatus according to claim 1, wherein the portion of the first filter wheel and/or the portion of the respective second engagement level of the second filter wheel, the third filter wheel, the fourth filter wheel and/or optionally each of the further filter wheels has or have teeth in a region of 45% to 55%, in particular 48% to 52%, preferably 50%, of the respective outer circumference.

5. The filter changing apparatus according to claim 1, wherein the respective filter wheel has four receptacles, six receptacles and/or optionally an even number of further receptacles for one optical filter each.

6. The filter changing apparatus according to claim 1, wherein the drive unit has a single motor for driving the first filter wheel or a terminal filter wheel of the filter changing apparatus.

7. The filter changing apparatus according to claim 6, wherein the first filter wheel or the terminal filter wheel has a drive portion comprising teeth distributed completely over the respective outer circumference so that the first filter wheel or the terminal filter wheel can be driven via a drive gear by means of the one single motor.

8. The filter changing apparatus according to claim 6, wherein the first filter wheel or the terminal filter wheel has a shaft or a receptacle for a shaft along the first rotary axle for connection to the one single motor.

9. The filter changing apparatus according to claim 1, wherein the connecting wheels have a common second rotary axle.

10. The filter changing apparatus according to claim 1, wherein the first rotary axle and/or the second rotary axle is or are arranged on the base plate.

11. The filter changing apparatus according to claim 1, wherein the filter changing apparatus has a holding device for holding a non-rotating filter wheel in its position during driving.

12. A camera head for an endoscope, the camera head having an image sensor, an opening for receiving light of an image along an optical path and an optical lens system for focusing the light on the image sensor, wherein the camera head has at least one filter changing apparatus according to claim 1.

13. A retrofit kit for retrofitting a camera head and/or an endoscope, wherein the retrofit kit has at least one filter changing apparatus according to claim 1 so that the filter changing apparatus can be arranged between a proximal end of the endoscope and a distal end of the camera head.
